(19) United States
(12) Patent Application Publication
(10) Pub. No.: US 2025/0002506 A1
(43) Pub. Date: Jan. 2, 2025

(54) IMPROVED PROCESS FOR THE PREPARATION OF LURBINECTEDIN AND ITS MORPHS THEREOF

(71) Applicants: RK Pharma Inc., Piscataway, NJ (US); APICORE PHARMACEUTICALS PRIVATE LIMITED, Vadodara, Gujarat (IN)

(72) Inventors: Jayaraman KANNAPPAN, Vadodara (IN); Dixit PANCHAL, Vadodara (IN); Gaurav YADAV, Vadodara (IN)

(73) Assignees: RK Pharma Inc., Piscataway, NJ (US); APICORE PHARMACEUTICALS PRIVATE LIMITED, Vadodara, Gujarat (IN)

(21) Appl. No.: 18/722,583

(22) PCT Filed: Sep. 30, 2022
(86) PCT No.: PCT/IB2022/059343

§ 371 Date: Jun. 21, 2024

(30) Foreign Application Priority Data
Nov. 15, 2021 (IN) 202141052219
Jan. 14, 2022 (IN) 202241002229
May 5, 2022 (IN) 202221026266

(51) Int. Cl. C07D 515/22

(52) U.S. Cl. CPC C07D 515/22

(57) ABSTRACT

The present invention provides unimproved synthetic process for the preparation of Lurbinectedin. The present invention also relates to new intermediates used in the preparation of Lurbinectedin. The present invention further provides new polymorphic form RK-1 of Lurbinectedin and process for producing the same. An improved process for the preparation of amorphous form of Lurbinectedin is also provided.

### RELATED APPLICATION

This application is a PCT International application and claims priority to and the benefit of Indian Provisional Patent Application Number 202141052219 dated 15 November 2021 and claims the priority to and the benefit of Indian Patent Application Number 202241002229 dated 14 Jan. 2022 and claims the priority to and the benefit of Indian Provisional Patent Application Number 202221026266 dated 5 May 2022 the contents of which are incorporated herein in its entirety.

### FIELD OF THE INVENTION

The present invention provides an improved synthetic process for the preparation of Lurbinectedin. The present invention also relates to new intermediate used in the preparation of Lurbinectedin. The present invention further provides new polymorphic form RK-1 of Lurbinectedin and process for producing the same. Additionally, an improved process for the preparation of an amorphous form of Lurbinectedin is also provided.

### BACKGROUND OF THE INVENTION

Lurbinectedin is a synthetic tetrahydropyrrolo [4, 3, 2-de]quinolin-8(JH)-one alkaloid analogue with potential antineoplastic activity. Lurbinectedin covalently binds to residues lying in the minor groove of DNA, which may result in delayed progression through S phase, cell cycle arrest in the G2/M phase and cell death.

Lurbinectedin, also known as PM01183 and initially called tryptamicidin, is a synthetic antitumoral compound that is currently in clinical trials for the treatment of cancer. The chemical structure of Lurbinectedin is represented by Formula 1.

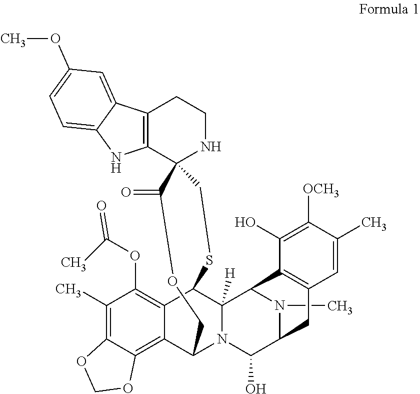

WO 03/014127 discloses Lurbinectedin, and a pharmaceutical composition containing Lurbinectedin. The process disclosed in the PCT application '127 patent involve preparation of Lurbinectedin by Scheme-I given below,

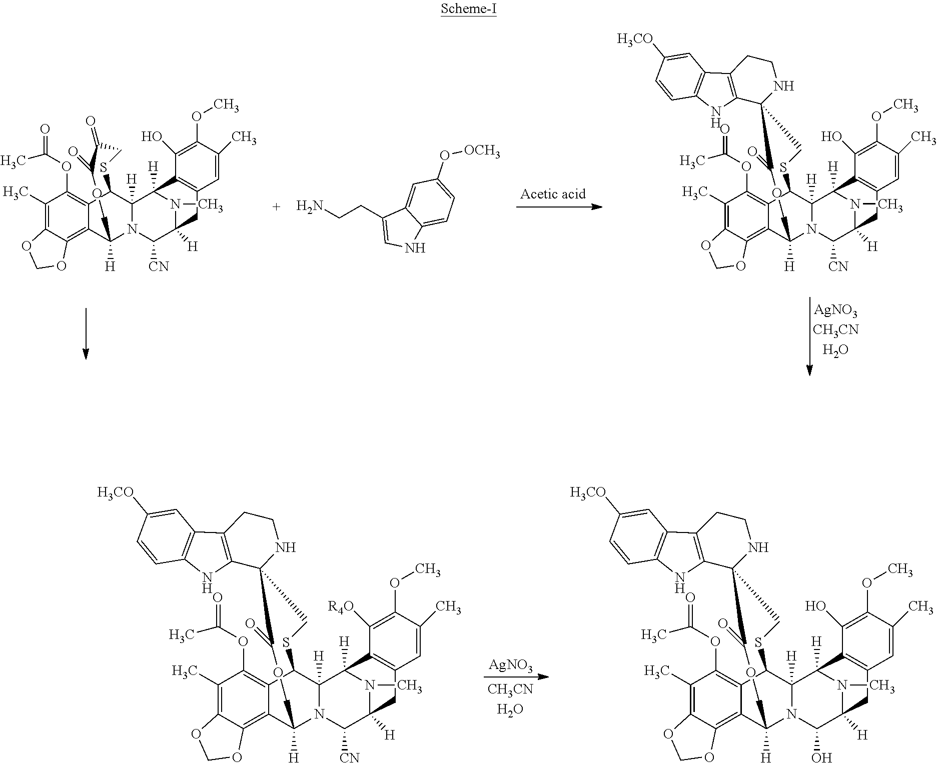

There is a need to develop viable, economical, simple and eco-friendly process for the reparation of Lurbinectedin.

The inventors of the present invention have developed an improved process for the preparation of Lurbinectedin surprisingly which is simple and circumvent the problems in the prior art processes and with which Lurbinectedin is obtained in good yield, with high purity, and with a controlled impurity content. The above said process is also surprisingly scalable at pivotal sizes and with a controlled impurity content.

Our process involves formation of methoxymethyl ether intermediate of compound of Formula-IV from Diketone intermediate of compound of Formula-III followed by conversion to in-situ intermediate of compound of formula-IVa and later conversion to Lurbinectedin and provides Lurbinectedin which is 99.7-99.9% pure.

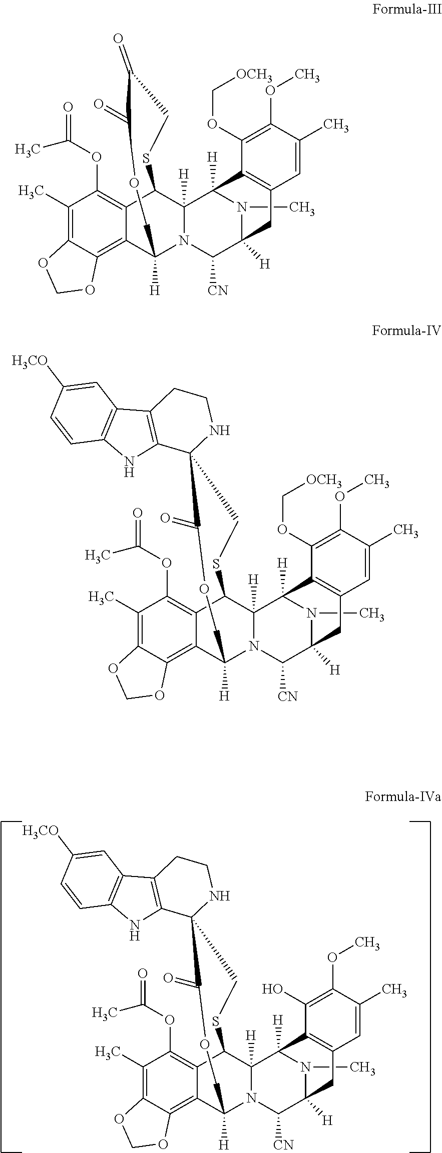

The form of Lurbinectedin obtained by the process described in WO 03/014127 is form A which is an amorphous form. However, process for the preparation of amorphous form of Lurbinectedin disclosed in the impugned application '4127 results in Lurbinectedin having low yield and with only 84.9% purity. Our invention provides pure substantially amorphous form of Lurbinectedin having purity greater than about 99.6%, preferably greater than 99.8% and wherein content of one or more impurities selected from in-situ intermediate impurity E, Deacetyl impurity F and Dehydroxy impurity G is less than 0.15%.

WO2021/099635 discloses Form B of Lurbinectedin. It further discusses process for the preparation of form B of Lurbinectedin as well as pharmaceutical composition comprising a form B of Lurbinectedin.

The present invention further provides new, stable and highly pure Polymorphic form RK-1 of Lurbinectedin and also provides process for its preparation. Polymorphism is a phenomenon relating to the occurrence of different crystal forms for one molecule. A single molecule, like Lurbinectedin, may give rise to variety of crystalline forms having distinct crystal structures and physical properties like melting point, X-ray diffraction pattern, infra absorption fingerprint, and solid state NMR spectrum. Different salts and solid state forms (including solvated forms) of an active pharmaceutical ingredient may possess different properties. Such variations in the properties of different salts and solid state forms and solvates may provide basis for improving formulation; for example, facilitating better processing or handling characteristics, improving the dissolution profile, or improving stability (polymorph as well as chemical stability) and shelf-life. Since improved drug formulations are consistently sought, there is an ongoing need for new, stable and pure polymorphic forms of existing drug molecules. For at least these reasons, there is a need for additional solid state form of Lurbinectedin.

### Advantages of the Present Invention

Simple, cost effective and eco-friendly process for the preparation of pure Lurbinectedin.
Pure Lurbinectedin obtained by the present invention is substantially free from Impurity-E, Impurity-F (Deacetyl imp) and Impurity-G (Dehydroxy Imp.)
Pure Lurbinectedin obtained by the present invention is chemically stable for a period of about 6 months under long term and accelerated storage conditions.
Lurbinectedin is obtained in good yield and the process allows manufacturing on a commercial scale with a low expenditure.
Invention provides an improved process for the preparation of substantially amorphous form of Lurbinectedin. having purity greater than about 99.6%, preferably greater than 99.8% and wherein content of one or more impurities selected from in-situ intermediate impurity E, Deacetyl impurity F and Dehydroxy impurity G is less than 0.15%.
Present invention further provides new, stable and highly pure polymorphic form RK-1 of Lurbinectedin and also provides process for its preparation.

### SUMMARY OF THE INVENTION

An embodiment of the present invention provides Pure Lurbinectedin having purity greater than about 99.6%, preferably greater than 99.8% which is substantially free from one or more impurities selected from in-situ impurity E, Deacetyl impurity F and Dehydroxy impurity G.

Another embodiment of the present invention provides a new and improved process for the preparation of Lurbinectedin, comprising the steps of:
i) Deallylation of compound of Formula-I to obtain compound of Formula-II;

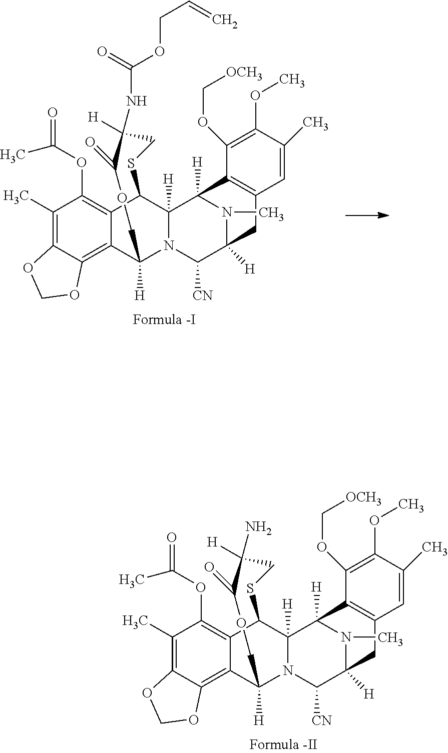

ii) Oxidation of amine group of compound of Formula-11 to corresponding alpha-keto lactone group by transamination to form compound of Formula-III.

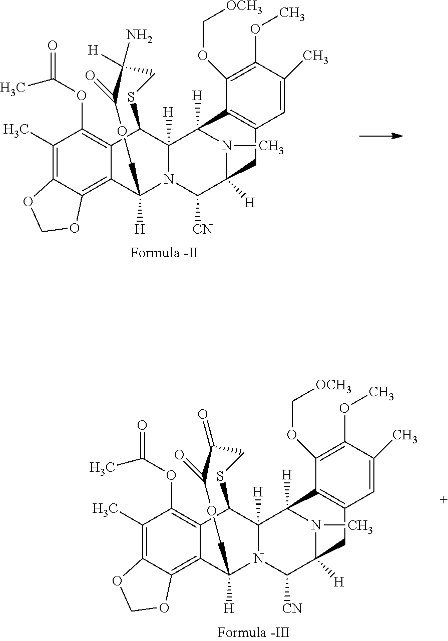

iii) Coupling of compound of Formula-III with 5-methoxytryptamine to obtain compound of Formula-IV.

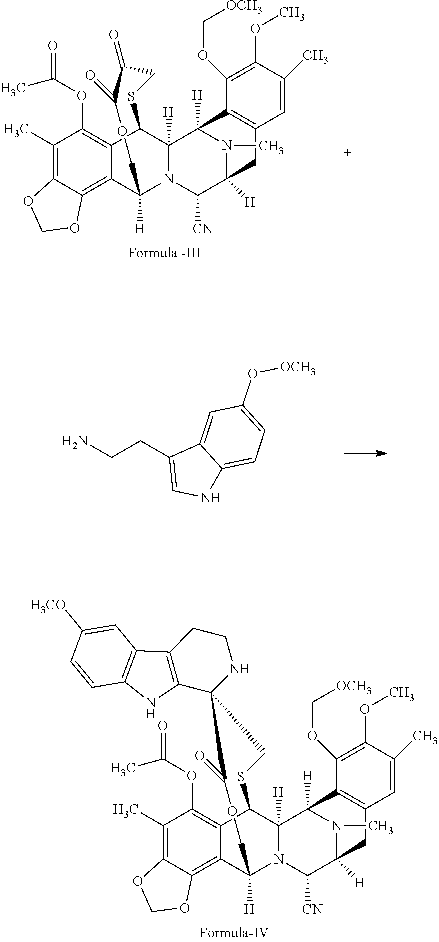

iv) Cleavage of methoxymethyl group of compound of Formula-IV to form compound of Formula-IVa in-situ, followed by hydrolysis of compound of Formula-IVa to obtain crude Lurbinectedin of formula V

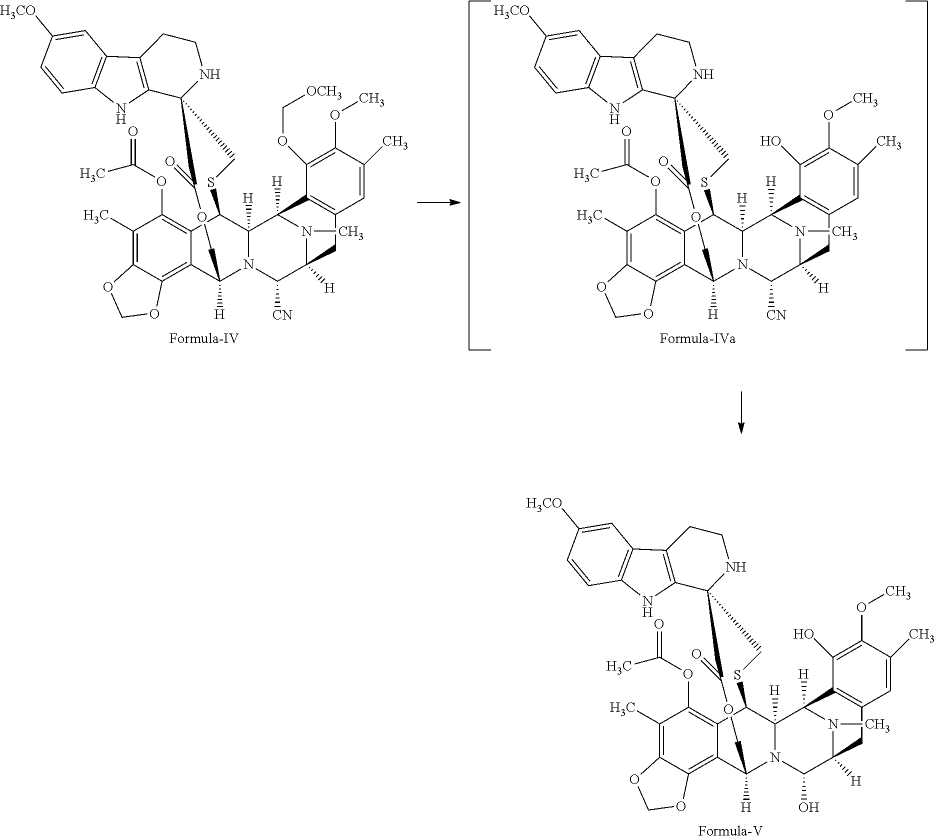

v) Purification of crude Lurbinectedin to obtain pure Lurbinectedin of Formula-V.

In a further embodiment, the invention also provides new intermediate compound of formula VI.

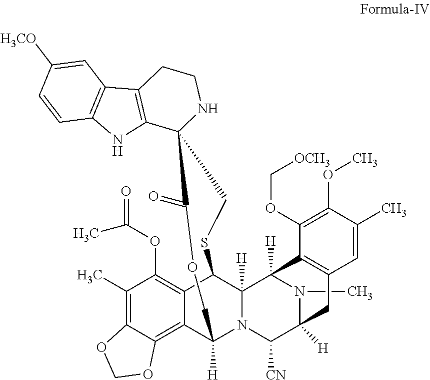 [SMILES: COCOc1c(OC)c(C)cc2c1[C@@H]1[C@@H]3[C@@H]4SC[C@]5(NCCc6c5[nH]c5ccc(OC)cc65)C(=O)OC[C@@H](c5c6c(c(C)c(OC(C)=O)c54)OCO6)N3[C@@H](C#N)[C@H](C2)N1C]

Another embodiment of the present invention provides an improved process for the preparation of substantially amorphous form of Lurbinectedin preferably having purity greater than about 99.80% characterized by PXRD pattern as illustrated in FIG. 1 and DSC pattern as illustrated in FIG. 2.
Further embodiment of the present invention provides new, stable and highly pure polymorphic form RK-1 of Lurbinectedin that can be characterized by its PXRD pattern as illustrated in FIG. 3 and DSC pattern as illustrated in FIG. 4 and also provides process for preparing the same.

### BRIEF DESCRIPTION OF THE DRAWINGS

FIG. 1: Characteristic X-ray powder diffraction pattern of amorphous form of Lurbinectedin

FIG. 2: Characteristic DSC of amorphous form of Lurbinectedin

FIG. 3: Characteristic X-ray powder diffraction pattern of polymorphic form RK-1 of Lurbinectedin obtained as per the Example 6

FIG. 4: Characteristic DSC of polymorphic form RK-1 of Lurbinectedin obtained as per the example 6

FIG. 5: Characteristic X-ray powder diffraction pattern of amorphous form of Lurbinectedin after storing for period of 1 month

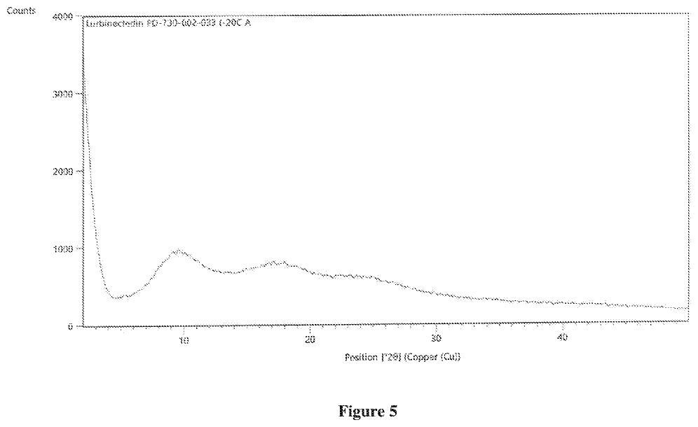

FIG. 6: Characteristic X-ray powder diffraction pattern of polymorphic form RK-1 of Lurbinectedin after storing for period of 1 month

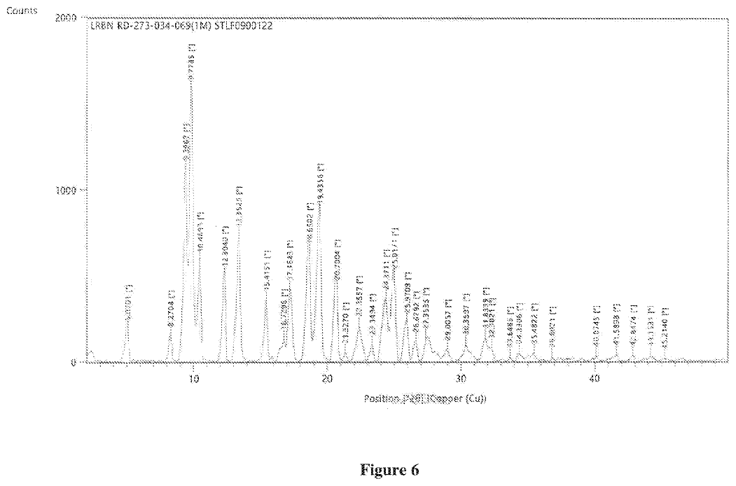

FIG. 7: Characteristic DSC of polymorphic form RK-1 of Lurbinectedin after storing for the period of 1 month

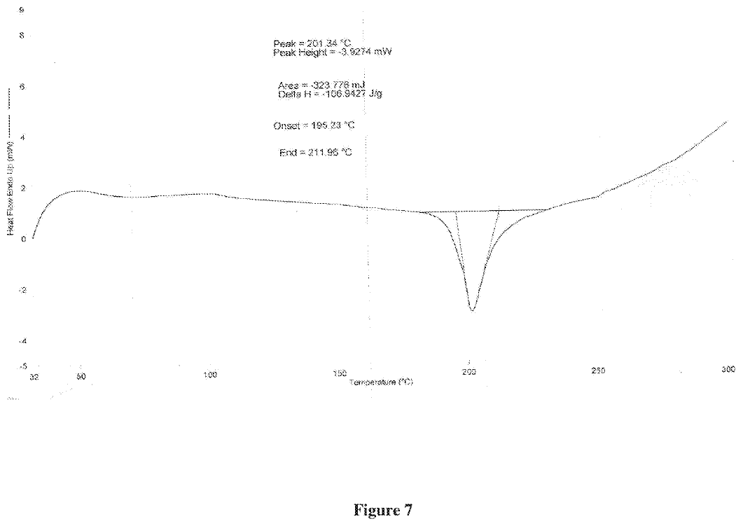

FIG. 8: Characteristic X-ray powder diffraction pattern of polymorphic form RK-1 of Lurbinectedin after storing for period of 3 months

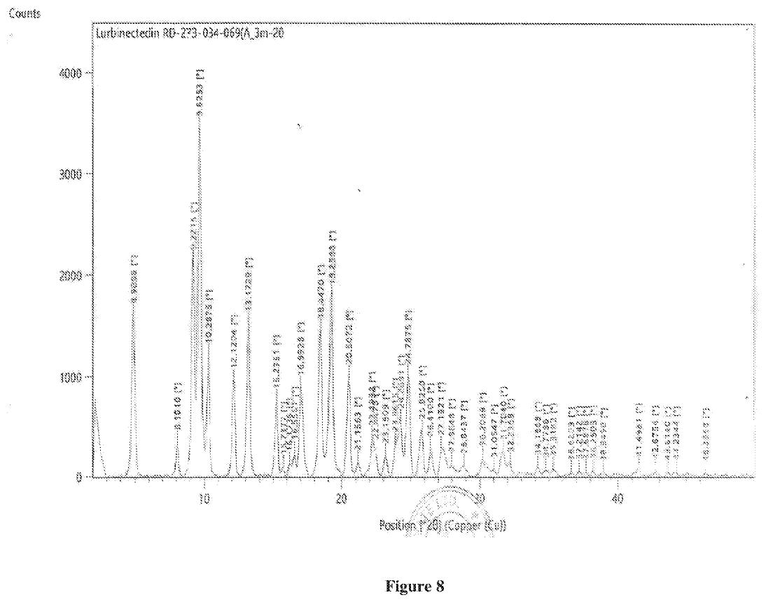

FIG. 9: Characteristic DSC of polymorphic form RK-1 of Lurbinectedin after storing for the period of 3 months

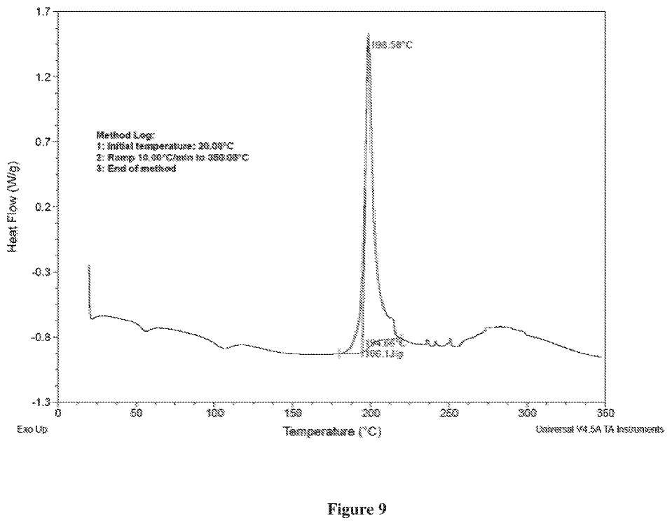

FIG. 10: Characteristic X-ray powder diffraction pattern of polymorphic form RK-1 of Lurbinectedin obtained as per the Example 7

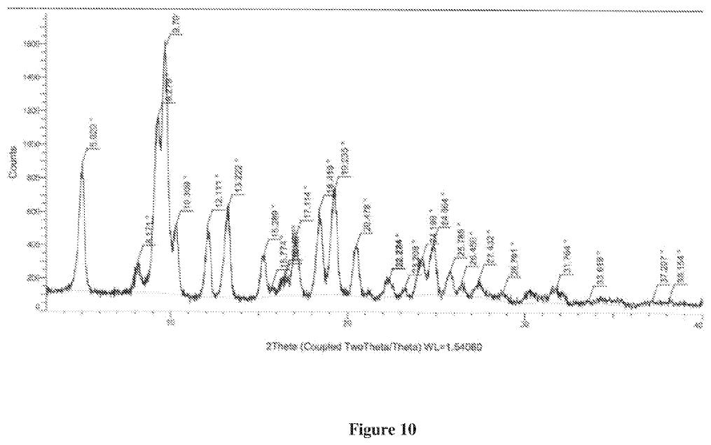

### DETAILED DESCRIPTION OF THE INVENTION

As used herein the term “pure” refers to Lurbinectedin having purity greater than about 99.6% or preferably greater than about 99.70% or more preferably greater than about 99.80% by high performance liquid chromatography (HPLC).

As used herein, the term “substantially free” refers to a compound of the present invention having one or more impurities less than 0.5% or less than 0.4% or less than 0.3% or less than 0.2% or less than 0.1% or less than 0.05% or less than about 0.03 or less than 0.01%; preferably less than 0.15%, more preferably less than 0.1%.

First embodiment of the present invention is to provide new and improved process for the preparation of Lurbinectedin, comprising the steps of:
i) Deallylation of compound of Formula-I in presence of reagent, solvent and optionally in presence of catalyst to obtain compound of Formula-II;

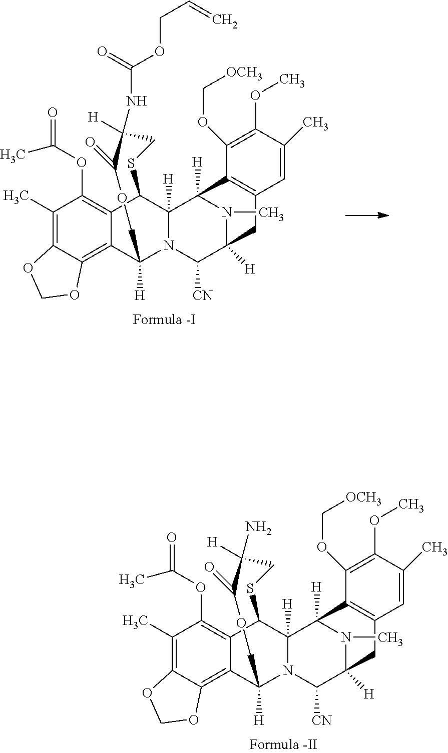

ii) Oxidation of amine group of compound of Formula-II to corresponding alpha-keto lactone group by transamination in presence of reagent, solvent and optionally in presence of catalyst to form compound of Formula-III;

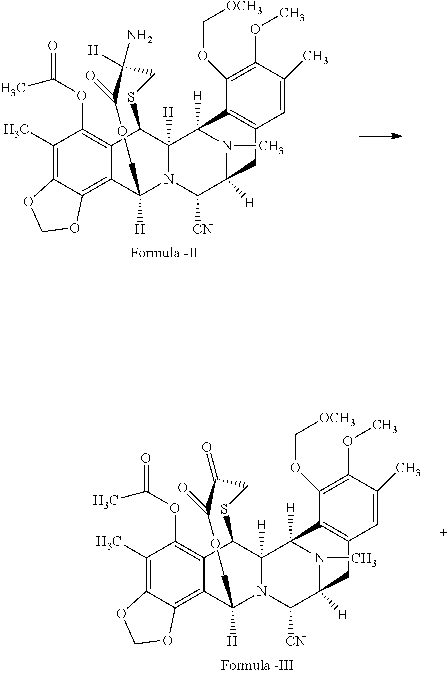

iii) Coupling of compound of Formula-III with 5-methoxytryptamine in presence of reagent, solvent and optionally in presence of catalyst to obtain compound of Formula-IV;

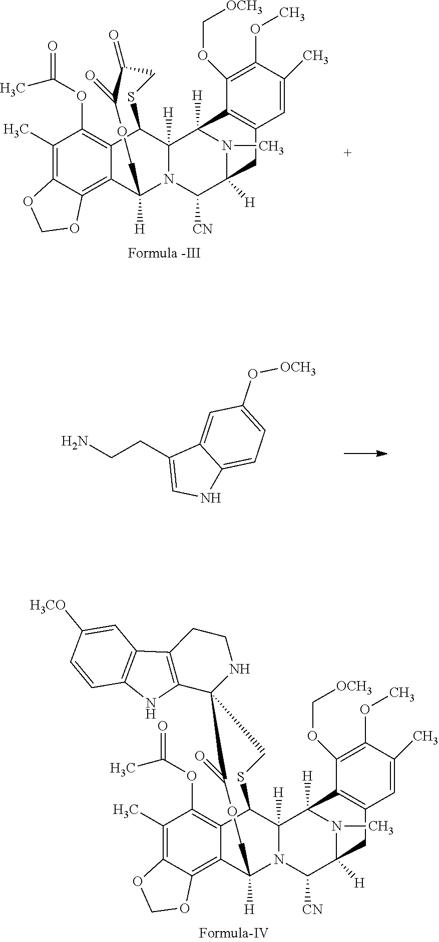

iv) Cleavage of methoxymethyl group of compound of Formula-IV to form compound of Formula-IVa in-situ in presence of reagent, solvent and optionally in presence of catalyst, followed by hydrolysis of compound of Formula-IVa in presence of reagent and solvent to obtain crude Lurbinectedin of Formula-V;

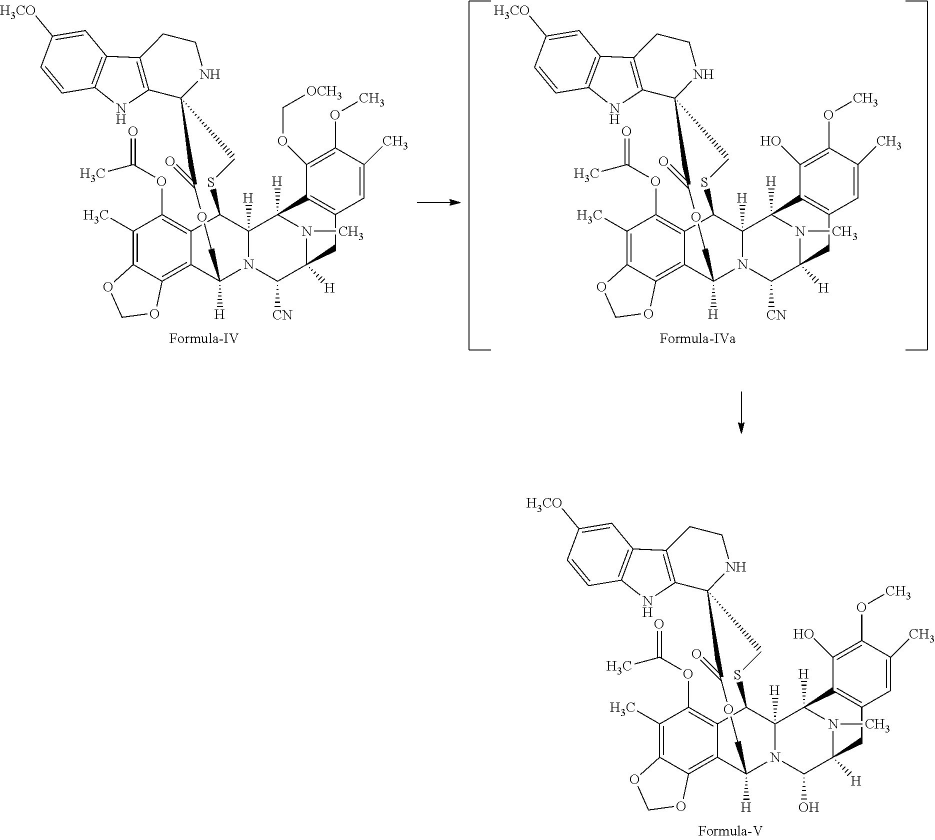

v) Purification of crude Lurbinectedin to obtain pure Lurbinectedin of Formula-V.
   According to second embodiment of the invention, catalyst used in step i) may be selected from Tributylstannane and Dichlorobis(triphenylphosphine)palladium; formic acid and palladium; or acetic acid and zinc; preferably, Tributylstannane and Dichlorobis(triphenyl phosphine)palladium.

According to another embodiment of the invention, reagent used in step i) may be selected from organic acid such as formic acid or acetic acid, preferably acetic acid.

According to further embodiment of the invention, solvent used in step i) may be selected from chlorinated solvent such as dichloromethane, dichloroethane and chlorobenzene or mixtures thereof; preferably, dichloromethane.

According to preferred embodiment of the invention, reaction of step i) is carried out in presence of Tributylstannane, acetic acid, Dichlorobis (triphenyl phosphine) palladium and dichloromethane.

According to further preferred embodiment of the invention, reaction of step i) is carried out at the temperature of −5° C. to −20° C., preferably −5° C. to −10° C. and under nitrogen atmosphere.

According to third embodiment of the invention, reagent used in step ii) may be selected from zinc sulfate and sodium glyoxylate/magnesium glyoxylate or 4-formyl-1-methylpyridinium benzenesulfonate or its monohydrate salt; preferably, 4-formyl-1-methylpyridinium benzenesulfonate monohydrate.

According to another embodiment of the invention, catalyst used in step ii) may be selected from 1,8-Diazabicyclo[5.4.0]undec-7-ene (DBU) and DABCO; preferably 1,8-Diazabicyclo[5.4.0]undec-7-ene (DBU).

According to further embodiment of the invention, solvent used in step ii) may be selected from water; nitrile solvents such as acetonitrile; amide solvents such as dimethyl formamide, dimethyl acetamide; chlorinated solvents such as dichloromethane, dichloroethane and chlorobenzene or mixtures thereof; preferably, mixture of dimethyl formamide and dichloromethane.

According to preferred embodiment of the invention, reaction of step ii) is carried out in presence of 4-formyl-1-methylpyridinium benzenesulfonate monohydrate, 1,8-Diazabicyclo[5.4.0]undec-7-ene (DBU), dimethyl formamide, dichloromethane and 4 Å activated molecular sieves.

According to further preferred embodiment of the invention, reaction of step ii), step iii) and step iv) is carried out at temperature of 20-40° C., preferably 20-30° C. under nitrogen atmosphere.

According to another preferred embodiment of the invention, reaction of step ii) and step iii) is carried out in presence of 4 Å activated molecular sieves.

According to fourth embodiment of the invention, reagent used in step iii) may be selected from silica; organic acid such as formic acid, acetic acid; acetate such as sodium acetate, potassium acetate; preferably, acetic acid.

According to another embodiment of the invention, solvent used in step iii) may be selected from water; alcohol such as methanol, ethanol, propanol, isopropanol; chlorinated solvents such as dichloromethane, dichloroethane and chlorobenzene or mixtures thereof; preferably dichloromethane or dichloroethane, more preferably dichloromethane.

According to preferred embodiment of the invention, reaction of step iii) is carried out in presence of acetic acid and dichloromethane.

According to fifth embodiment of the invention, reagent used for cleavage of methoxymethyl group of compound of Formula-IV in step iv) may be selected from acid such as trifluoroacetic acid, hydrochloric acid, sulfuric acid; gas such as hydrogen; trialkylsilyl chloride such as trimethylsilyl chloride; preferably, trimethylsilyl chloride.

According to another embodiment of the invention, solvent used for cleavage of methoxymethyl group of compound of Formula-IV in step iv) may be selected from water, alcohol such as methanol, ethanol, propanol, isopropanol; ester such as ethyl acetate; ether such as tetrahydrofuran, dimethyl ether, diethyl ether; nitrile solvents such as acetonitrile; amide solvents such as dimethyl formamide, dimethyl acetamide; chlorinated solvents such as dichloromethane, dichloroethane and chlorobenzene or mixtures thereof; preferably, mixture of acetonitrile and dichloromethane.

According to further embodiment of the invention, catalyst used for cleavage of methoxymethyl group of compound of Formula-TV in step iv) may be selected from acid such as acetic acid; sodium catalyst such as sodium acetate, sodium bisulfate, sodium iodide; palladium catalyst such as palladium, palladium hydroxide, palladium diacetate; lithium catalyst such as lithium chloride, lithium iodide, lithium hydroxide; copper catalyst such as cupric chloride, cupric acetate; preferably, sodium iodide

According to preferred embodiment of the invention, cleavage of methoxymethyl group of compound of Formula-IV in step iv) is performed in presence of trimethylsilyl chloride, sodium iodide, acetonitrile and dichloromethane.

According to sixth embodiment of the invention, reagent used for hydrolysis of compound of Formula-IVa in step iv) may be selected from silver nitrate.

According to another embodiment of the invention, solvent used for hydrolysis of compound of Formula-IVa in step iv) may be selected from water; alcohol such as methanol, ethanol, propanol, isopropanol; ester such as ethyl acetate; ether such as tetrahydrofuran, dimethyl ether, diethyl ether; nitrile solvents such as acetonitrile; amide solvents such as dimethyl formamide, dimethyl acetamide; chlorinated solvents such as dichloromethane, dichloroethane and chlorobenzene or mixtures thereof; preferably, mixture of water and acetonitrile, more preferably mixture of deoxygenated water and deoxygenated acetonitrile.

According to preferred embodiment of the invention, hydrolysis of compound of Formula-IVa in step iv) is performed in presence of silver nitrate, deoxygenated water and deoxygenated acetonitrile.

According to seventh embodiment of the invention, crude Lurbinectedin obtained in step iv) may be further purified by conventional purification techniques, preferably by preparative chromatography using methanol and water as solvents in presence of ammonium acetate and acetic acid; followed by lyophilization to obtain pure Lurbinectedin.

According to preferred embodiment of the invention, pure Lurbinectedin obtained after step v) is in amorphous form.

According to another embodiment of the invention, pure Lurbinectedin obtained after step v) is having purity of greater than 99.6%, preferably greater than 99.8%.

According to further embodiment of the invention, pure Lurbinectedin obtained after step v) contains less than 0.5% or less than 0.4% or less than 0.3% or less than 0.2% or less than 0.1% or less than 0.05% or less than 0.03 or less than 0.01%; preferably less than 0.15%, more preferably less than 0.1% of one or more impurities selected from in-situ intermediate impurity E, Deacetyl impurity F and Dehydroxy impurity G

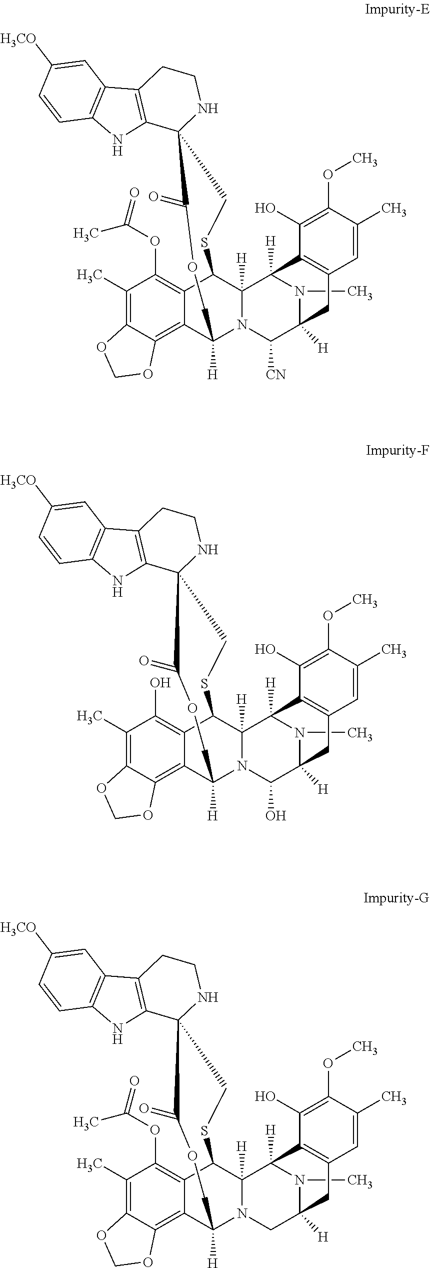 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1C#N)N2C)OCO4 ; COc1ccc2[nH]c3c(c2c1)CCN[C@]31CS[C@@H]2c3c(O)c(C)c4c(c3[C@H](COC1=O)N1[C@@H]2[C@H]2c3c(cc(C)c(OC)c3O)C[C@@H]([C@@H]1O)N2C)OCO4 ; COc1ccc2[nH]c3c(c2c1)CCN[C@]31CS[C@@H]2c3c(OC(C)=O)c(C)c4c(c3[C@H](COC1=O)N1C[C@@H]3Cc5cc(C)c(OC)c(O)c5[C@H]([C@H]21)N3C)OCO4]

According to eighth embodiment, the invention provides new intermediate of compound of formula VI and process for its preparation.

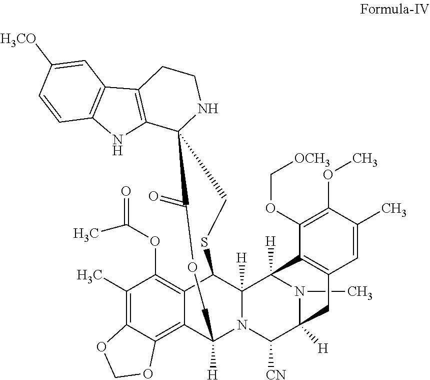 [SMILES: COCOc1c(OC)c(C)cc2c1[C@@H]1[C@@H]3[C@@H]4SC[C@]5(NCCc6c5[nH]c5ccc(OC)cc65)C(=O)OC[C@@H](c5c6c(c(C)c(OC(C)=O)c54)OCO6)N3[C@@H](C#N)[C@H](C2)N1C]

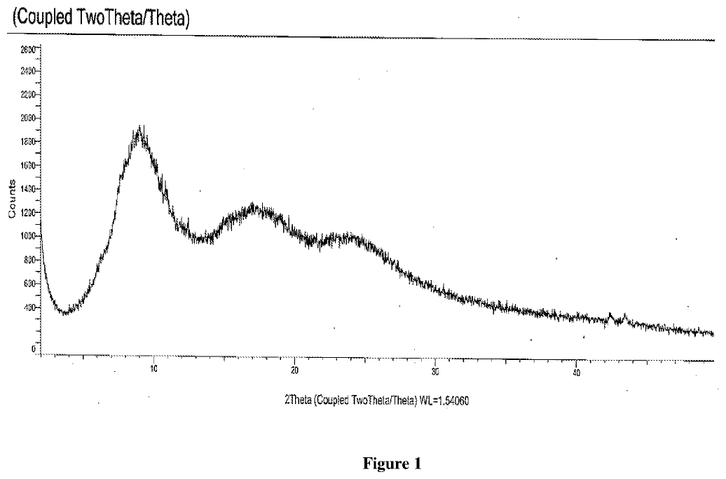

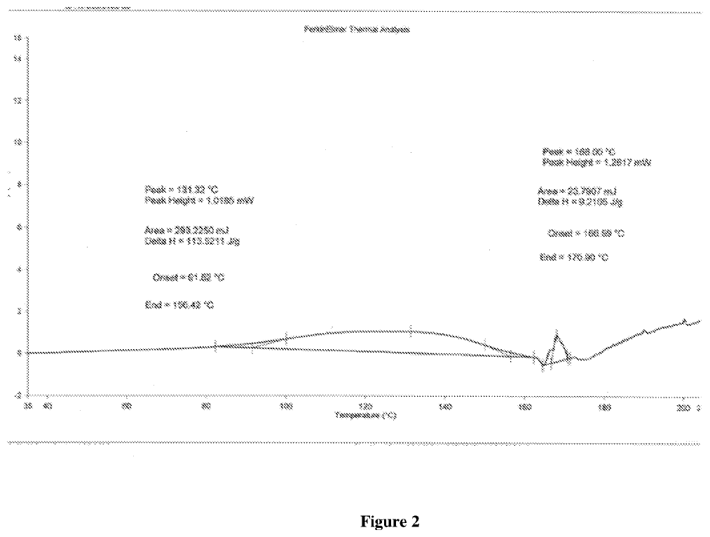

According to ninth embodiment, present invention provides an improved process for the preparation of a substantially amorphous form of Lurbinectedin characterized by PXRD pattern as illustrated in FIG. 1 and DSC pattern as illustrated in FIG. 2, having purity greater than 99.6%, preferably greater than 99.8%, wherein content of one or more impurities selected from in-situ intermediate impurity E, Deacetyl impurity F and Dehydroxy impurity G is less than 0.15%, comprising the steps of:
a) Preparatory purification of crude Lurbinectedin by HPLC using alcohol and water solvent mixture
b) Final isolation of pure amorphous form of Lurbinectedin by lyophilization.

According to preferred embodiment, alcohol used in step a) is methanol.

According to tenth embodiment, present invention provides substantially amorphous form of Lurbinectedin characterized by PXRD pattern as illustrated in FIG. 1 and DSC pattern as illustrated in FIG. 2, having purity of greater than 99.6%, preferably greater than 99.8%, wherein content of one or more impurities selected from in-situ intermediate impurity E, Deacetyl impurity F and Dehydroxy impurity G is less than 0.15%.

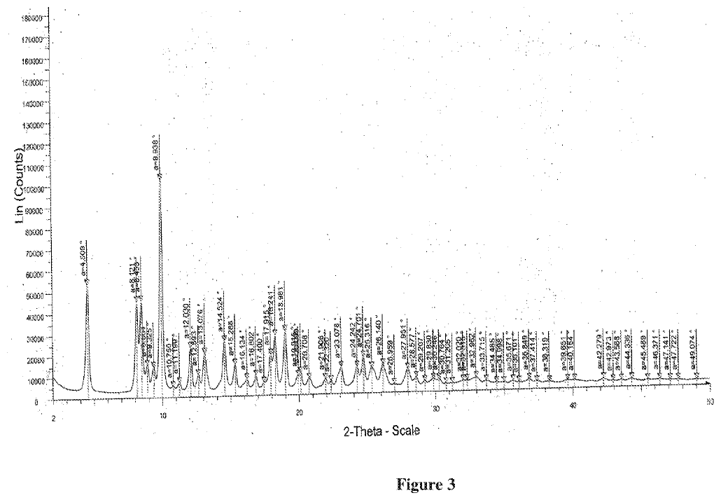

According to eleventh embodiment, present invention provides new, stable and highly pure polymorphic form RK-1 of Lurbinectedin, characterized by a powder X-ray diffraction pattern as illustrated by FIG. 3 comprising at least seven, preferably at least five or more characteristic crystalline peaks expressed in degrees 2-theta selected from 4.5±0.2°, 5.0±0.2°, 8.1±0.2°, 8.5±0.2°, 9.8±0.2°, 13.1±0.2°, 17.2±0.2°, 24.4±0.2°, 25.9±0.2.

According to another embodiment of the invention, RK-1 Polymorphic form of Lurbinectedin further comprises peaks at 2-theta angles selected from 9.2±0.2°, 12.0±0.2°, 15.2±0.2°, 18.4±0.2°, 19.2±0.2°, 20.6±0.2°, 24.9±0.2°, 26.45±0.2°.

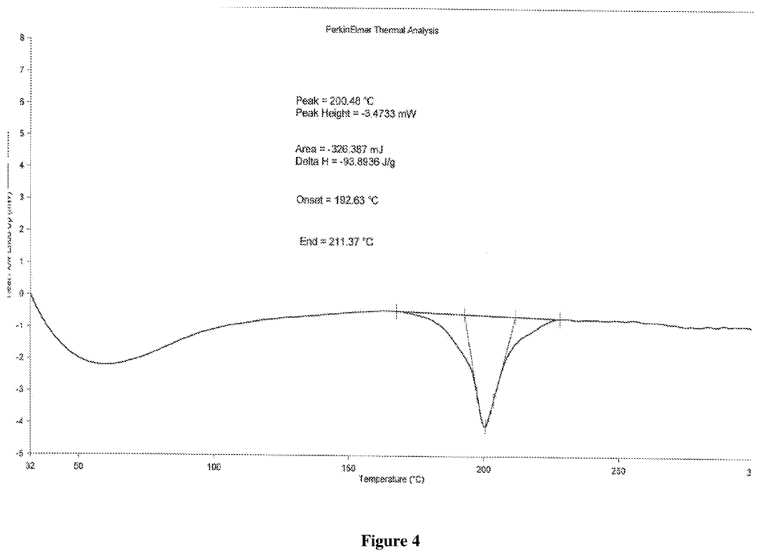

According to preferred embodiment, polymorphic form RK-1 of Lurbinectedin can be characterized by DSC showing characteristic peak at 200.48° C. An illustrative DSC thermogram of polymorphic form RK1 of Lurbinectedin has been shown in FIG. 4.

According to further preferred embodiment of the invention, RK-1 polymorphic form of Lurbinectedin is having purity greater than about 99.6% or preferably greater than about 99.70% or more preferably greater than about 99.80% by high performance liquid chromatography (HPLC); wherein content of one or more impurities less than 0.5% or less than 0.4% or less than 0.3% or less than 0.2% or less than 0.1% or less than 0.05% or less than about 0.03 or less than 0.01%; preferably less than 0.15%, more preferably less than 0.1%.

According to twelfth embodiment of the invention, present invention also provides process for the preparation of polymorphic form RK-1 polymorphic form of Lurbinectedin, characterized by a powder X-ray diffraction pattern as illustrated by FIG. 1 comprising a characteristic crystalline peaks expressed in degrees 2-theta selected from 4.5±0.2°, 5.0±0.2°, 8.1±0.2°, 8.5±0.2°, 9.8±0.2°, 13.1±0.2°, 17.2±0.2θ, 24.4±0.2°, 25.9±0.2 comprising the steps of:
a) Dissolution of Lurbinectedin in suitable polar solvent;
b) Precipitation of Polymorphic form RK-1 of Lurbinectedin by addition of suitable anti-solvent into the polar solvent specified in step a) given above or vice versa to obtain polymorphic form RK-1 of Lurbinectedin.

According to preferred embodiment of the invention, polar solvent used in step a) includes chlorinated hydrocarbon such as methylene dichloride (MDC), carbon tetrachloride, chloroform or 1, 2-dichloroethane; ester such as ethyl acetate, isopropyl acetate, butyl acetate, isobutyl acetate, methylbutyryl acetate, ethylbutyryl acetate or Methyl isobutyrylacetate; alcohol such as methanol, ethanol, n-propanol or isopropanol; ketone such as acetone, dimethyl ketone, diethyl ketone or methyl ethyl ketone; nitrile such as acetonitrile, propionitrile, butyronitrile or isobutyronitrile; amide such as dimethylformamide (DMF), dimethylacetamide or diethyl acetamide; sulfone such as dimelthylsulfoxide (DMSO), or diethyl sulfone or mixture thereof.

According to further preferred embodiment of the invention, anti-solvent used in step b) includes but not limited to hydrocarbon such as n-pentane, n-hexane, n-heptane, n-octane. cyclohexane, benzene or toluene, chlorinated hydrocarbon such as dichloromethane, chloroform, carbon tetrachloride and 1,2-dichloroethane, ether such as dimethyl ether, diethyl ether, di-isopropyl ether, methyl tert-butyl ether, tetrahydrofuran or 1,4-dioxane or mixture thereof.

According to most preferred embodiment of the invention, polar solvent used in step a) is chlorinated hydrocarbon selected from dichloromethane, chloroform, carbon tetrachloride and 1,2-dichloroethane or mixtures thereof; preferably dichloromethane.

According to another most preferred embodiment of the invention, anti-solvent used in step b) is alkane selected from n-pentane, n-hexane, n-heptane and n-octane; preferably n-heptane.

According to further preferred embodiment of the invention, Lurbinectedin employed in step a) is amorphous polymorphic form of Lurbinectedin.

The present invention will be further illustrated with reference to the following examples which aid in the understanding, but which are not to be construed as limitations thereof.

### EXAMPLES

### Example 1: Preparation of Compound of Formula-II from Compound of Formula-I

Four-neck 5000 ml round bottom flask was arranged under nitrogen atmosphere with thermometer pocket and overhead stirrer. Dichloromethane (1250 ml) was charged into round bottom flask at 20 to 30° C. 50 gm of Compound of Formula-I and 1250 ml of Dichloromethane were added at 20 to 30° C. The reaction mass was stirred for 5 to 10 minutes at 20 to 30° C. Dichlorobis triphenylphosphine (Pd) (0.46 g) was added into the reaction mixture at 20 to 30° C. and reaction mass was stirred for 5 to 10 min at 20 to 30° C. The reaction mass was cooled at −10 to −5° C. and acetic acid (19.9 g) was added into the reaction mixture at −5 to −10° C. Tributyl tin hydride (193 g) was slowly added into the reaction mixture at −10 to −5° C. and reaction mass was stirred for 25 to 35 min at −10 to −5° C. 500 ml of water was added into a separate 5.0 Liter RBF and cooled to 0 to 10° C. The reaction mass was slowly added lot wise into it at 0 to 10° C. and RBF was washed with 2×100 ml of dichloromethane. 10% sodium bicarbonate solution was added into the reaction mass at 0 to 10° C. to adjust pH up to 7.0 to 8.80. The reaction mass was stirred for 10 min. at 0 to 10° C. and layers were separated. Aqueous layer was extracted with dichloromethane at 5° C. to 15° C. multiple number of times. The Organic layers were combined and solvent was distilled out under vacuum at not more than 35° C. and the product was degassed under vacuum for 30 min. at not more than 35° C.

The reaction mass was cooled to 20 to 30° C. and acetonitrile 500 mL was charged into RB flask at 20° C. to 30° C. The reaction mass was stirred for 10 minutes at 20 to 30° C. and n-hexane 1000 mL was charged into RB flask at 20 to 30° C. The reaction mass was stirred for 10 minutes at 20 to 30° C. The acetonitrile & hexane layers were separated and the acetonitrile layer was extracted with hexane multiple number of times. Acetonitrile layers were combined and solvents were distilled under vacuum at not more than 40° C. 250 ml of ethyl acetate was added and solvents were distilled under vacuum at not more than 40° C. The product was degassed under vacuum for 30-60 min. at not more than 40° C. The reaction mass was cooled to 20 to 30° C. and ethyl acetate 50 mL was charged into RB flask at 20° C. to 30° C. under nitrogen atmosphere. The reaction mass was stirred for 15 minutes at 20° C. to 30° C. and 1000 ml Hexane was charged into RB flask at 20° C. to 30° C. The reaction mass was stirred for 60 minutes at 20° C. to 30° C. and the product was filtered under nitrogen atmosphere and vacuum and then washed with hexane (250 ml) and suck dried well.

### Example 2: Preparation of Compound of Formula-III from Compound of Formula-II

50 L 4-necked round bottom flask was arranged under nitrogen atmosphere with thermometer pocket and overhead stirrer. 125 g of 4-Formyl-1-methylpyridinium benzenesulfonate monohydrate and N, N-dimethylformamide (2000 ml) were charged at 20 to 30° C. in nitrogen atmosphere and the reaction mass was stirred for 15 minutes at 20 to 30° C. Dichloromethane (400 ml) was added lot-wise into reaction mass and 240 g of activated 4 A° molecular sieve was added at 20° C. to 30° C. and then reaction mass was stirred for 15 minutes at 20° C. to 30° C. 40 g of Product of Example 1 (compound of Formula-II) and 1200 ml of dichloromethane were added into reaction mass at 20° C. to 30° C. and the reaction mass was stirred for 5 to 6 hrs. at 20° C. to 30° C. 1, 8-Diazabicyclo (5.4.0) undec-7-ene (22 g) was slowly added and the funnel was flushed with 20 ml of dichloromethane into the reaction mixture at at 20° C. to 30° C. The reaction mass was stirred for 15-25 minutes at 20 to 30° C. and cooled to 0 to 5° C. Freshly prepared 10% citric acid solution (1200 ml) was added into reaction mixture at 0 to 5° C. and the temperature of the reaction mixture was raised to 20 to 30° C. The reaction mass was stirred for 25-45 minutes at 20 to 30° C. 2800 ml of process water was charged into reaction mass at 20 to 30° C. and the reaction mass was stirred for 10 minutes at 20 to 30° C. 400 ml dichloromethane was added into the reaction mass and reaction mass was stirred for 10-15 minutes at 20° C. to 30° C. The reaction mass was filtered through celite bed and the bed was washed with 600 ml of dichloromethane at 20-30° C. Filtrate ml was charged into separating funnel and the reaction mass was allowed to settle for not less than 10 minutes. Layers were separated, aqueous layer was charged back into RB flask and 600 ml dichloromethane was added into RB flask at 20° C. to 30° C. The reaction mass was stirred for 10 to 15 minutes at 20° C. to 30° C., allowed to settle and layers were again separated. Aqueous layer was charge back into RB flask and 2400 ml of MTBE was added into reaction mass at 20 to 30° C. and the reaction mass was stirred for 15 minutes at 20 to 30° C. The layers were separated and aqueous layer was extracted with MTBE multiple number of times. Organic layers were combined and pH of the reaction mass was adjusted to 8.00-9.50 by addition of 2.5% sodium bicarbonate solutions at 20° C. to 30° C. Layers were separated and aqueous layer was again extracted with MTBE multiple number of times. All organic layers were combined, 200 g sodium sulphate was added and the reaction mass was stirred for 10 to 15 minutes. The reaction mass was settled for not less than 10 minutes, organic layer was decanted and sodium sulphate was washed with 400 ml MTBE at 20° C. to 30° C. Solvent was distilled under vacuum at not more than 40° C. and the product was degassed under vacuum for 30 to 60 min at not more than 40° C. The content was cooled to 20° C. to 30° C., product was transferred into 3.0 lit RB flask and cooled at 15° C. to 20° C. 600 ml MTBE was charged into RB flask at 15° C. to 20° C. The reaction mass was stirred for 10 minutes to 15 minutes at 15° C. to 20° C. and 1200 mL process water was charged into RB flask at 15° C. to 20° C.

Reaction mass was stirred for 10 minutes to 15 minutes, allowed to settle and layers were separated. Organic layer was charged back into RB flask, 1200 ml of process water was added into reaction mass at 15° C. to 20° C. and the reaction mass was stirred for 10 minutes to 15 minutes. Reaction mass was allowed to settle, layers were separated and aqueous layer was extracted with MTBE multiple number of times. All organic layers were combined, 120 g sodium sulphate was added and the reaction mass was stirred for 10 to 15 minutes. Reaction mass was allowed to settle for not less than 10 minutes, organic layer was decanted and sodium sulphate was washed by 400 ml MTBE at 20° C. to 30° C. Solvents were distilled under vacuum at water bath temperature not more than 40° C. and the product was degassed under vacuum for not less than 30 min at water bath temperature not more than 42° C. 400 ml of n-Hexane was charged and the reaction mass was cooled at 20° C. to 30° C. Reaction mass was stirred for 60 to 70 min at 20 to 30° C. The product was then filtered under nitrogen atmosphere and under vacuum, washed with 250 ml hexane and suck dried well. The solid was dried under vacuum for 6.0 hrs at 30° C. to 35° C.2.5 volume of methanol and Dried Ketone compound of formula III obtained above were charged in RB flask at 20° C.-30° C. The reaction mass was stirred for 5 to 10 min at 20° C. to 30° C. and the temperature of the reaction mass was raised up to 45° C. to 50° C. Reaction mass was stirred for 30 to 35 min, heating was stopped and the reaction mass was gradually cooled up to 20° C. to 30° C. Reaction mass was stirred for 55 to 65 min, filtered through buckner funnel and washed with 1.50 volume of methanol. Obtained solid was dried under vacuum for 6-7 hrs at 30° C. to 35° C. to obtain the titled compound.

### Example 3: Preparation of Compound of Formula IV from Compound of Formula-III

3000 ml 4-neck round bottom flask was arranged under nitrogen atmosphere with thermometer pocket and overhead stirrer. 14 gm of compound of Formula-III obtained in Example 2 and 140 ml dichloromethane were charged into RB flask at 20° C. to 30° C. under nitrogen atmosphere. The reaction mass was stirred for 10-15 minutes at 20° C. to 30° C. and 11.5 gm 5-Methoxytryptamine and 70 ml dichloromethane were charged into the reaction mixture at 20° C. to 30° C. under nitrogen atmosphere. 12.60 g acetic acid was charged into the reaction mass at 20° C. to 30° C. and reaction mass was stirred for 60 min to 70 min at 20° C. to 30° C. The reaction mass was cooled to 5° C. to 15° C. and pH of the reaction mass was adjusted to 7.0-9.3 by 10% sodium bicarbonate solutions at 5° C. to 15° C. The reaction mass was stirred for 5-10 minutes at 5° C. to 15° C., filtered through celite bed and the bed was washed with 56.0 mL dichloromethane.

Filtrate mL was charged into separating funnel. The reaction mass was allowed to settle for not less than ten minutes and layers were separated. Aqueous layer was extracted with dichloromethane multiple number of times.

The organic layers were combined, 0.7 gm of activated charcoal was added and reaction mass was stirred for 10 to 15 minutes at 20° C. to 30° C. The organic layer was filtered through celite bed and washed with 56 ml of dichloromethane. Filtrate ml and 42 g sodium sulphate were charged into round bottom flask and reaction mass was stirred for 10 to 15 minutes. The reaction mass was allowed to settle for not less than 10 minutes, organic layer was decanted and sodium sulphate was washed with 56 mL dichloromethane. The solvent was distilled under vacuum. The reaction mass was cooled to 20° C. to 30° C. and 70 ml ethyl acetate was added and the reaction mass was stirred for 10-15 minutes at 20° C. to 30° C. 350 ml n-hexane was added into RB flask at 20° C. to 30° C. and the reaction mass was stirred for 30-45 minutes at 20° C. to 30° C. The product was filtered under nitrogen atmosphere and vacuum, washed with n-hexane and product was then dried in vacuum Tray Drier at 25° C.-30° C. for 4 to 6 hours to obtain compound of formula IV.

### Example 4: Preparation of Crude Lurbinectedin of Compound of Formula-V from Compound of Formula-IV

Anhydrous Sodium iodide (13.8 g) and Acetonitrile (350 ml) were charged at 20° C. to 30° C. into a 3 Liter 4-neck round bottom flask equipped with nitrogen atmosphere, overhead stirrer and thermometer pocket. The reaction mass was stirred for 30 to 45 min. at 20° C. to 30° C. Dichloromethane (300 ml) was charged into reaction mass at 20° C. to 30° C. and Compound of Formula-III (15 g) obtained in Example 3 was added into reaction mass at 20° C. to 30° C. The reaction mass was stirred for 10-15 minutes at 20° C. to 30° C. and cooled to −10° to −15° C. Chlorotrimethyl silane (10.05 g) was slowly added into reaction mass at −10° to −15° C. and the reaction mass was stirred for 90 min. at −10° to −15° C. Temperature of the reaction mixture was raised to 0° to 5° C. and 300 ml of dichloromethane was added into reaction mixture at 0 to 5° C. 1000 ml of 10% sodium dithionate solution was added into reaction mixture at 0 to 5° C. and the temperature of the reaction mass was raised to 20° C. to 30° C. The reaction mass was stirred for 10-15 minutes at 20° C. to 30° C., reaction mass was allowed to settle and the layers were separated. Organic layer was charged back into RBF and 1000 ml of 10% sodium dithionate solution was added at 20° C. to 30° C. The reaction mass was stirred for 15 minutes at 20° C. to 30° C., reaction mass was allowed to settle and the layers were separated. The aqueous layers were combined and extracted with dichloromethane multiple number of times. The organic layers were then combined, 225 ml of 5.0% sodium bicarbonate solution was added into the RB flask at 20° C. to 30° C. and the reaction mass was stirred for 10 to 15 min. Reaction mass was allowed to settle, layers were separated and aqueous layer was extracted with dichloromethane multiple number of times. Organic layers were combined, 30 g sodium sulphate was added and the reaction mass was stirred for 10 to 15 minutes. Reaction mass was allowed to settle, organic layer was decanted and sodium sulphate was washed by 450 ml dichloromethane. Solvent was distilled out under vacuum at not more than 40° C. and the product was degassed under vacuum.

The reaction mass was cooled to 20° C. to 30° C. and the obtained compound was dissolved in deoxygenated acetonitrile (1200 ml) into a 10 Liter 4-Neck round bottom flask. The reaction mass was stirred for 10-15 min. at 20° C. to 30° C. Deoxygenated (W.F.I) water (1800 ml) was charged into reaction mass and the reaction mass was stirred for 10-15 min. at 20° C. to 30° C. Silver nitrate (92.25 g) was added lot-wise into the reaction mass at 20° C. to 30° C. and the reaction mass was stirred for 5 to 6 hrs at 20° C. to 30° C. The reaction mixture was cooled to 0 to 10° C. and 450 ml 5% sodium bicarbonate solution was added into the reaction mass at 0 to 10° C. 900 ml of dichloromethane was added into reaction mixture at 0 to 10° C. followed by addition of 300 ml of process water. The temperature of the reaction mixture was raised to 10° C. to 15° C. and the reaction mass was stirred for 10-15 min at 10° C. to 15° C. The reaction mass was then filtered through celite bed at 10 to 15° C. under vacuum and washed with 600 ml of dichloromethane.

Filtered ML was charged back into RBF, layers were separated and aqueous layer was extracted with dichloromethane multiple number of times. The organic layers were combined and 3.0 gm activated carbon was added into the organic layer. Temperature of the reaction mixture was raised to 20-30° C. and the reaction mass was stirred for 10 to 15 min at 20° C. to 30° C. The reaction mass was filtered through celite bed at 20° C. to 30° C. under vacuum. Filtrate was collected, 30.0 g sodium sulphate was charged into it and reaction mixture was stirred for 10 to 15 min and allowed it to settle for some time. Organic layer was decanted, solvent was distilled out under vacuum and product was degassed for 30 to 60 min to obtain Dry weight B of compound of formula V. Crude Lurbinectedin was isolated using Method-I or Method-II mentioned below.

Method-I: Isolation of Crude Lurbinectedin Using Dichloromethane and n-Heptane

Dichloromethane (BX 40V) and the (Dry wt.-B) of the titled compound obtained above were charged into a round bottom flask. Solution was filtered through 0.45 micron filter and washed by the BX5V of Dichloromethane at 20 to 30° C. Filtered solution was added into 20 Liter Assembly containing BX400V of n-Heptane within 30 to 60 min. at 20 to 30° C. in nitrogen atmosphere and washed by BX2V of Dichloromethane at 20 to 30° C. The reaction mass was stirred for 30 to 60 min. at 20 to 30° C. in nitrogen atmosphere. The reaction mass was filtered and washed by BX5V of n-Heptane and the product was dried under vacuum to obtain crude Lurbinectedin of Formula-V.

Method-II: Isolation of Crude Lurbinectedin Using Ethyl Acetate and n-Hexane

Ethyl acetate (BX 10V) and the (Dry wt.-B) of the titled compound obtained above were charged into a round bottom flask. The reaction mass was stirred for 20 to 30 min. at 20° C. to 30° C. and (BX 12V) ml n-hexane was added in to the reaction mass at 20° C. to 30° C. The reaction mass was stirred 60 to 70 min. at 20° C. to 30° C. The product was filtered, washed with (BX 10V) n-hexane and the product was dried under vacuum to obtain crude Lurbinectedin of Formula-V.

### Example 5: Preparation of Amorphous Form of Lurbinectedin

Crude Lurbinectedin obtained in Example 4 was loaded lot wise and the preparative HPLC was run under following chromatographic conditions:

### Chromatographic Conditions:

Wavelength: 210 nm
Flow Rate: 100 ml/min
Run time: about 240 mins
Diluent: 3% Acetic acid in MeOH:Water (98:2)
Retention time: about 100 mins
Preparation of Mobile phases:
Mobile phase A: 0.1% Ammonium acetate+0.1% Glacial acetic acid in Water
Mobile phase B: Methanol
Gradient Program:—Linear Gradient

At the time of Lurbinectedin main peak elution, eluent was collected and that pooled eluent fraction was analyzed by HPLC. The pooled fractions were mixed into a single container and proceeded for isolation by Lyophilization. The preparative fraction was stored at −20 to −40° C. Once preparative HPLC purification was completed, the prep fraction was filtered through 0.2° micron filter paper under nitrogen atmosphere at −10 to −20° C. Meanwhile the process water was cooled to below 5° C. in another flask. Above prechilled water was added into prep fraction for lyophilization process in Tray Lyophiliser.

All the trays were covered with perforated SS Tray guard. Lyophilization cycle was started. Once Lyophilization cycle was completed, the cycle was stopped and the vacuum was released. The trays were removed from Lyophiliser, immediately covered by using liner and after the temperature comes down to 20 to 25° C. the amorphous form of lurbinectedin was unloaded and stored at −2012° C. temperature. Amorphous form of Lurbinectedin was characterized by XRPD and DSC. Characteristic X-ray powder diffraction pattern and Differential Scanning Calorimetry chromatogram of amorphous form of Lurbinectedin are shown in FIG. 1 and FIG. 2 respectively.
Purity: 99.77%, Yield: 40 to 50% (w/w)

### Example 6: Preparation of RK-1 Polymorphic Form of Lurbinectedin

Lurbinectedin (Dry wt.-C) obtained in Example 5 and dichloromethane (C X 80 V) were charged into round bottom flask under nitrogen atmosphere at 20 to 30° C. Solution was filtered through 0.45 micron filter and washed by the suitable quantities of Dichloromethane at 20 to 30° C. Filtered solution was added into 20 Liter Assembly containing (C X 800 V) n-Heptane within 30 to 60 min. at 20 to 30° C. under nitrogen atmosphere. The reaction mass was stirred for 30 to 60 min. at 20 to 30° C., filtered and washed by suitable quantities of n-Heptane.

The material was suck dried for 30 to 60 min under nitrogen atmosphere, unloaded and charged into Lyophiliser to remove residual solvents. After reshuffling, the material was collected from all the trays and dried at −20 to −22° C. to obtain RK-1 polymorphic form of Lurbinectedin. Form RK-1 of Lurbinectedin was characterized by XRPD and DSC. Characteristic X-ray powder diffraction pattern and Differential Scanning Calorimetry chromatogram of RK-1 polymorphic form of Lurbinectedin are shown in FIG. 3 and FIG. 4 respectively. Endotherm of RK-1 polymorphic form of Lurbinectedin obtained by the technique of DSC starts at 170° C. and ends up to 210° C. in comparison with innovator's From B which starts at 130° C. and ends at 170° C.
Yield: 92%, Purity: 99.68%

### Example 7: Preparation of RK-1 Polymorphic Form of Lurbinectedin

Lurbinectedin (Dry wt.-C) obtained in Example 5 and dichloromethane (C X 80 V) were charged into round bottom flask under nitrogen atmosphere at 20 to 30° C. Solution was filtered through 0.45 micron filter and washed by the suitable quantities of Dichloromethane at 20 to 30° C. Filtered solution was added into 20 Liter Assembly containing (C X 800 V) n-pentane within 30 to 60 min. at 20 to 30° C. under nitrogen atmosphere. The reaction mass was stirred for 30 to 60 min. at 20 to 30° C., filtered and washed by suitable quantities of n-pentane.

The material was suck dried for 30 to 60 min under nitrogen atmosphere, unloaded and charged into Lyophiliser to remove residual solvents. After reshuffling, the material was collected from all the trays and dried at −20 to −22° C. to obtain RK-1 polymorphic form of Lurbinectedin. Form RK-1 of Lurbinectedin was characterized by XRPD and DSC. Characteristic X-ray powder diffraction pattern and Differential Scanning Calorimetry chromatogram of RK-1 polymorphic form of Lurbinectedin are shown in FIG. 3 and FIG. 4 respectively.
Yield: 89%, Purity: 99.72%

### Example 8: Preparation of RK-1 Polymorphic Form of Lurbinectedin

Lurbinectedin (Dry wt.-C) obtained in Example 5 and Carbon tetrachloride (C X 80 V) were charged into round bottom flask under nitrogen atmosphere at 20 to 30° C. Solution was filtered through 0.45 micron filter and washed by the suitable quantities of Carbon tetrachloride at 20 to 30° C. Filtered solution was added into 20 Liter Assembly containing (C X 800 V) n-heptane within 30 to 60 min. at 20 to 30° C. under nitrogen atmosphere. The reaction mass was stirred for 30 to 60 min. at 20 to 30° C., filtered and washed by suitable quantities of n-heptane.

The material was suck dried for 30 to 60 min under nitrogen atmosphere, unloaded and charged into Lyophiliser to remove residual solvents. After reshuffling, the material was collected from all the trays and dried at −20 to −22° C. to obtain RK-1 polymorphic form of Lurbinectedin. Form RK-1 of Lurbinectedin was characterized by XRPD and DSC. Characteristic X-ray powder diffraction pattern and Differential Scanning Calorimetry chromatogram of RK-1 polymorphic form of Lurbinectedin are shown in FIG. 3 and FIG. 4 respectively.
Yield: 90%, Purity: 99.68%

### Example 9: Preparation of RK-1 Polymorphic Form of Lurbinectedin

RK-1 polymorphic form of Lurbinectedin was synthesized by method similar to example 6 using following combination of solvents.

| Solvent 1 | Solvent 2 |
|---|---|
| Carbon tetrachloride | N-pentane |
| Chloroform: Carbon tetrachloride (1:1) | N-hexane: N-heptane (1:1) |
| Chloroform | N-hexane |
| 1, 2-dichloroethane | N-octane |

### Stability Study

An appropriate amount of the sample of the amorphous as well as RK-1 polymorphic form of Lurbinectedin obtained in examples 5 to 9 was placed in a reagent bottle at-20° C. under a sealed condition for the period of three months to perform a stability test. X-ray powder diffraction pattern, HPLC purity as well as DSC Chromatogram of amorphous as well as RK-1 polymorphic form of Lurbinectedin was evaluated again after storing for period of 3 months.

Result of the stability study of both amorphous (Obtained in Example 5) as well as RK-1 polymorphic form of Lurbinectedin (Obtained in Example 6) has been tabulated in Tables 1-2 given below.

TABLE 1
Stability study of Amorphous form of Lurbinectedin

| Initial | | At 3 Months | |
|---|---|---|---|
| | Off white | | Off white |
| Description | powder | Description | powder |
| Water content | 9.67% | Water content | 9.91% |
| HPLC purity | | | |
| RRT | Initial | RRT | A/3M |
| LRBN | 99.81% | LRBN | 99.77% |
| 0.06 | — | 0.07 | 0.06 |
| 0.20 | 0.02 | 0.20 | 0.02 |
| 0.85 | — | 0.85 | 0.01 |
| 0.98 | 0.03 | 0.98 | 0.03 |
| 1.02 | — | 1.02 | 0.05 |
| 1.07 | 0.02 | 1.07 | 0.02 |
| 1.17 | 0.04 | 1.17 | — |
| 1.23 | 0.03 | 1.23 | — |
| 1.31 | 0.02 | 1.31 | 0.02 |
| 1.38 | — | 1.38 | 0.01 |
| 1.41 | — | 1.41 | 0.01 |

TABLE 2
Stability study of the polymorphic form RK-1 of Lurbinectedin

| Initial | | At 3Months | |
|---|---|---|---|
| | Off white | | Off white |
| Description | powder | Description | powder |
| Water content | 9.69% | Water content | 10.1% |

HPLC purity

| | | | |
|---|---|---|---|
| RRT | Initial | RRT | A/3M |
| LRBN | 99.86% | LRBN | 99.77% |
| 0.07 | — | 0.07 | 0.06 |
| 0.20 | 0.03 | 0.20 | 0.02 |
| 0.85 | — | 0.85 | 0.01 |
| 0.98 | 0.03 | 0.98 | 0.03 |
| 1.02 | — | 1.02 | 0.05 |
| 1.07 | 0.02 | 1.07 | 0.02 |
| 1.17 | 0.04 | 1.17 | — |
| 1.23 | 0.03 | 1.23 | — |
| 1.31 | 0.02 | 1.31 | 0.02 |
| 1.38 | — | 1.38 | 0.01 |
| 1.41 | — | 1.41 | 0.01 |

Result of stability study confirmed that the impugned invention provides substantially pure Amorphous as well as polymorphic form RK-1 of Lurbinectedin having purity greater than 99.7% wherein content of one or more impurities selected from in-situ intermediate impurity E, Deacetyl impurity F and Dehydroxy impurity G is less than 0.15%.

The characteristic X-ray powder diffraction pattern of amorphous as well as RK-1 polymorphic form of Lurbinectedin obtained after storing for the period of three months was found to be the same as obtained initially which confirmed that amorphous as well as RK-1 polymorphic form of Lurbinectedin are stable on storage. Characteristic peaks in the XRPD pattern and DSC chromatogram of RK-1 polymorphic form of Lurbinectedin obtained initially as well as after storing for the period of three months have been tabulated in Table 3 given below.

TABLE 3
Characteristic peaks in XRPD and DSC of RK-1 polymorphic form of
Lurbinectedin initially and after storing for the period up to 3 months

| Parameter | Initial | After 1 month | After 3 months |
|---|---|---|---|
| Description | Off white powder | Off white powder | Off white powder |
| DSC Peak | 200.48 | 201.34 | 198.58 |
| 2-theta values | 5.03 | 5.07 | 4.90 |
| | 9.36 | 9.39 | 9.22 |
| | 9.81 | 9.77 | 9.62 |
| | 10.42 | 10.46 | 10.3 |
| | 12.25 | 12.30 | 12.12 |
| | 13.36 | 13.35 | 13.17 |
| | 15.43 | 15.41 | 15.23 |
| | 17.18 | 17.16 | 17.0 |
| | 18.61 | 18.65 | 18.44 |
| | 19.45 | 19.43 | 19.25 |
| | 20.65 | 20.70 | 20.50 |
| | 24.34 | 24.37 | 24.26 |
| | 25.0 | 25.0 | 24.8 |

Commercial advantages of the present invention
over the marketed preparation.

| | Cost per g raw | Lyophilization | Overall |
|---|---|---|---|
| Preparations | material materials | cost | cost |
| Marketed preparation | $ 30000 | $5000 | $ 35000 |
| Present invention | $15000 | $ 1000 | $ 16000 |

# Commercial advantages of the present invention over
the prior arts in terms of yield

| Preparations | Yield | Purity |
|---|---|---|
| Comparator-Patent US7763615 | <50% | 84.9% |
| Present invention | 50-75% | >99.60% |

An embodiment of the improved process has resulted in controlled formation of impurities and thus resulting in the final compound having high degree of purity.

Following Process related and degradation impurities were controlled in Lurbinectedin API obtained according to the present invention up to the limit of not more than 0.15%.

| Impurity Name | Structure |
|---|---|
| Impurity-A (Lactone) | 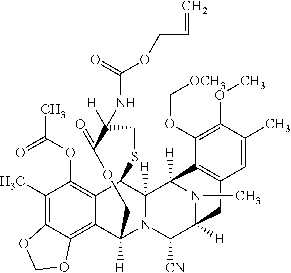 |
| Impurity-B (Amine) | 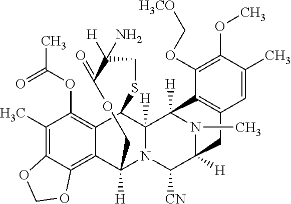 |
| Impurity-C (Ketone) | 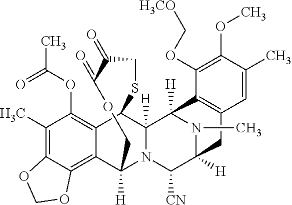 |
| Impurity-D (Methoxy methyl ether) | 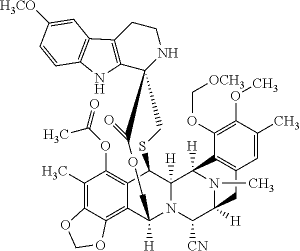 [SMILES: COCOc1c(OC)c(C)cc2c1[C@@H]1[C@@H]3[C@@H]4SC[C@]5(NCCc6c5[nH]c5ccc(OC)cc65)C(=O)OC[C@H](c5c6c(c(C)c(OC(C)=O)c54)OCO6)N3[C@@H](C#N)[C@H](C2)N1C] |
| Impurity-E (Dmethoxy methyl ether) | 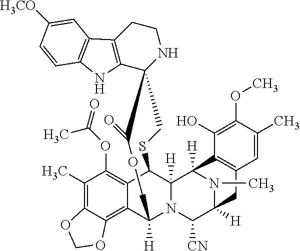 [SMILES: COc1ccc2[nH]c3c(c2c1)CCN[C@]31CS[C@@H]2c3c(OC(C)=O)c(C)c4c(c3[C@@H](COC1=O)N1[C@@H]2[C@H]2c3c(cc(C)c(OC)c3O)C[C@@H]([C@@H]1C#N)N2C)OCO4] |
| Impurity-F | |
| (Deacetyl imp.) | |
| Impurity-G | |
| (Dehydroxy | |
| Imp.) | |
| (5- Methoxy- tryptamine.) | 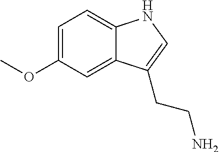 |

Without wishing to be bound to a theory, the process described in the present invention is believed to be an improved process for the preparation of Lurbinectedin which is commercially scalable, economical, stable and provides novel crystalline forms of Lurbinectedin with a characteristic XRPD pattern, and in some cases with a highly purified form of Lurbinectedin.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. Pure Lurbinectedin compound of Formula-V 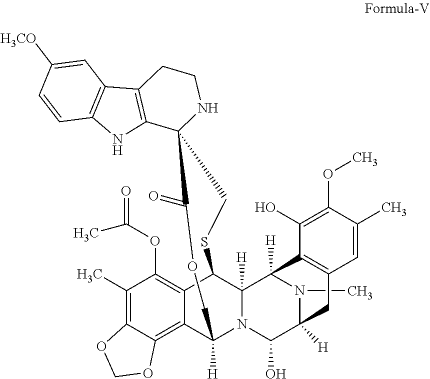 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]
having purity greater than 99.7% wherein content of one or more impurities selected from in-situ intermediate impurity E, Deacetyl impurity F and Dehydroxy impurity G is lower than 0.15%.

2. The pure Lurbinectedin as claimed in claim 1, wherein pure Lurbinectedin is amorphous polymorphic form of Lurbinectedin or RK-1 polymorphic form of Lurbinectedin of formula (V).

3. A process for the preparation of Pure Lurbinectedin of Formula-V, comprising the steps of:
i) deallylating compound of Formula-I in presence of reagent, solvent and optionally in presence of catalyst to obtain compound of Formula-II; 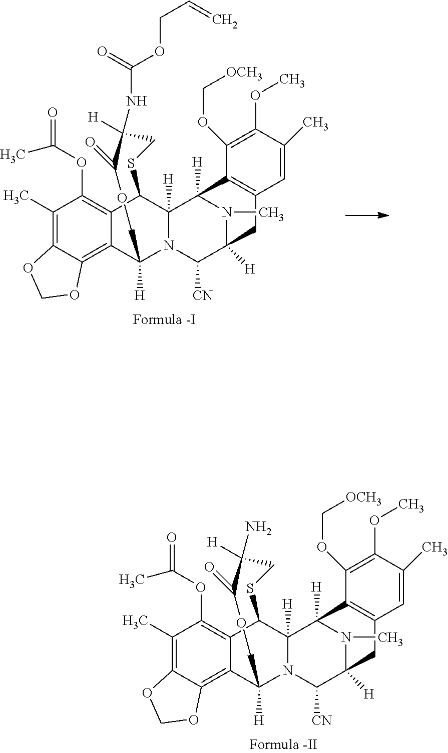
ii) oxidizing amine group of compound of Formula-II to corresponding alpha-keto lactone group by transamination in presence of reagent, solvent and optionally in presence of catalyst to form compound of Formula-III; 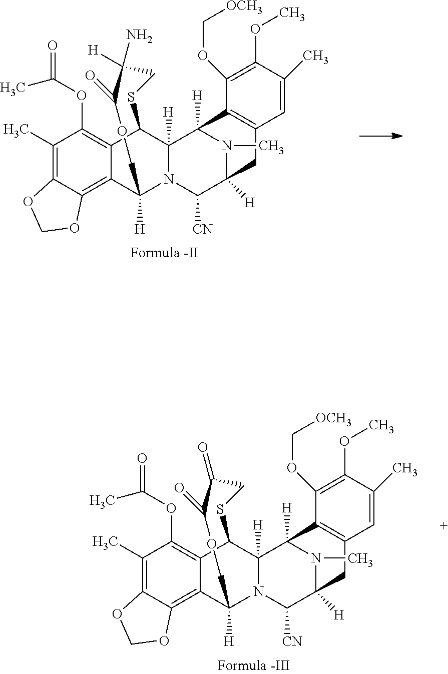
iii) coupling of compound of Formula-III with 5-methoxytryptamine in presence of reagent, solvent and optionally in presence of catalyst to obtain compound of Formula-IV; 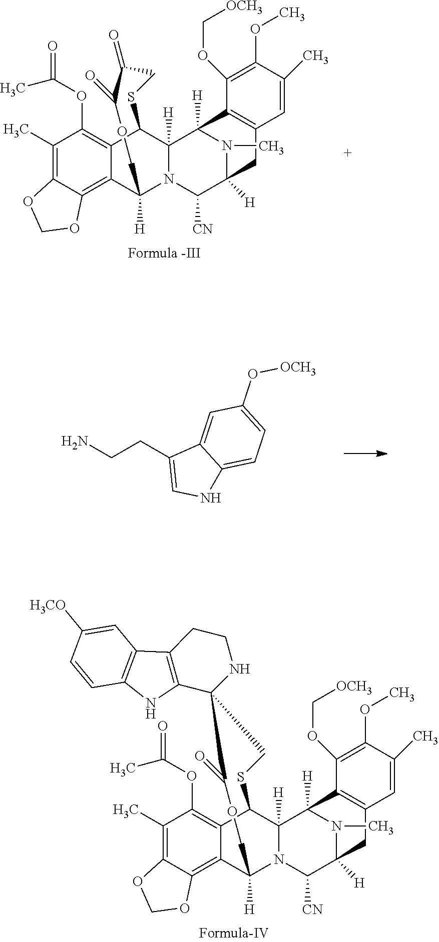
iv) cleaving of methoxymethyl group of compound of Formula-IV to form compound of Formula-IVa in-situ in presence of reagent, solvent and optionally in presence of catalyst, followed by hydrolysis of compound of Formula-IVa in presence of reagent and solvent to obtain crude Lurbinectedin of Formula-V; 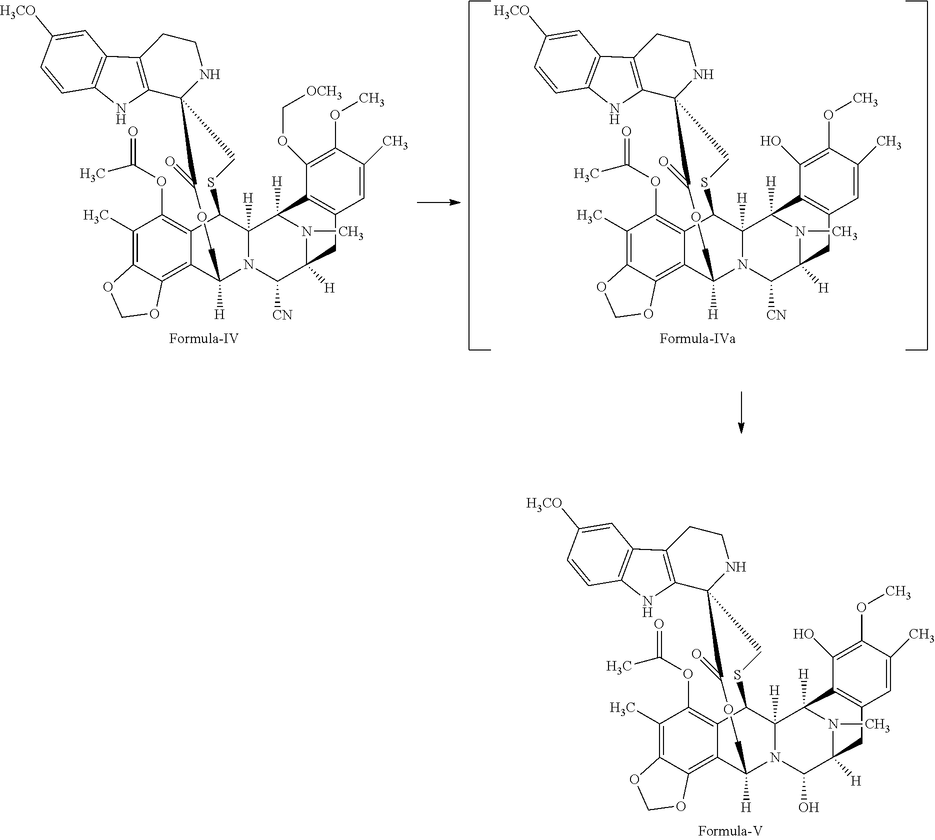
v) optionally purifying the crude Lurbinectedin by preparative HPLC using alcohol and water solvent mixture followed by lyophilization to obtain pure Lurbinectedin of formula V.

4. The process as claimed in claim 3, wherein pure Lurbinectedin is in amorphous form.

5. The process as claimed in claim 3, wherein, reaction of step i) is carried out in presence of tributylstannane, acetic acid, dichlorobis(triphenylphosphine)palladium and dichloromethane.

6. The process as claimed in claim 3, wherein, reaction of step ii) is carried out in presence of 4-formyl-1-methylpyridinium benzenesulfonate monohydrate, 1,8-diazabicyclo [5.4.0]undec-7-ene (DBU), dimethyl formamide, dichloromethane and 4 Å activated molecular sieves.

7. The process as claimed in claim 3, wherein, reaction of step iii) is carried out in presence of sodium acetate, ethanol, 4 Å activated molecular sieves.

8. The process as claimed in claim 3, wherein, cleavage of methoxymethyl group of compound of Formula-IV in step iv) is performed in presence of trimethylsilyl chloride, sodium iodide, acetonitrile and dichloromethane.

9. The process as claimed in claim 3, wherein, hydrolysis of compound of Formula-IVa in step iv) is performed in presence of silver nitrate, deoxygenated water and deoxygenated acetonitrile.

10. The process as claimed in claim 3, wherein alcohol used in step v) is methanol.

11. The process as claimed in claim 3, further comprises the step of preparation of compound of formula IV by coupling the compound of Formula-III with 5-methoxytryptamine in presence of suitable reagent and solvent and optionally in presence of catalyst to obtain compound of Formula-IV.

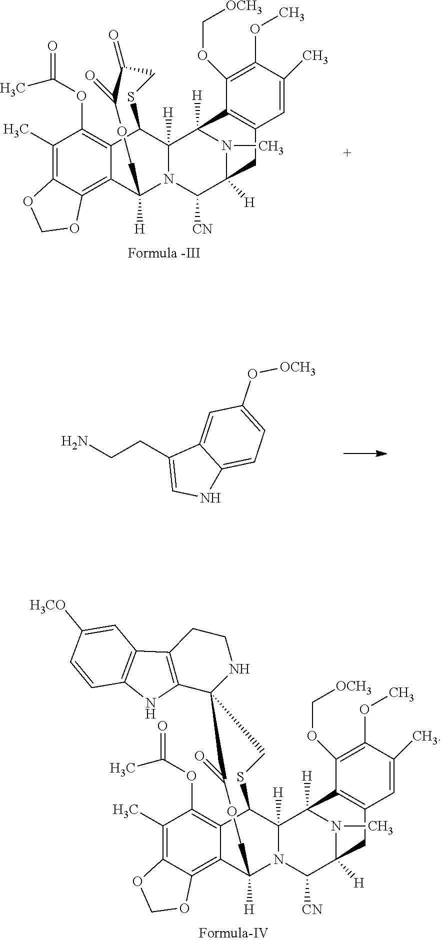

12. The pure Lurbinectedin as claimed in claim 2, wherein the RK-1 Polymorphic form of Lurbinectedin of formula (V), 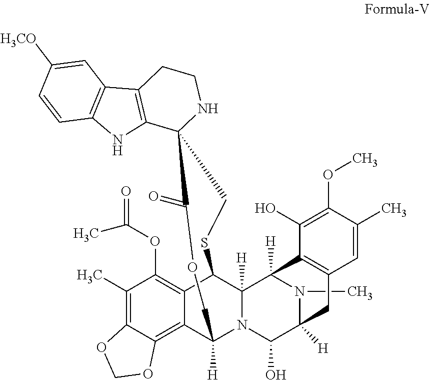 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]
is characterized by a powder X-ray diffraction pattern comprising at least five or more peaks at 2-theta angles selected from 4.5±0.2°, 5.0±0.2°, 8.1±0.2°, 8.5±0.2°, 9.8±0.2°, 13.1±0.2°, 17.2±0.2°, 24.4±0.2°, and 25.9±0.2°,

13. The pure Lurbinectedin as claimed in claim 12, wherein the RK-1 Polymorphic form of Lurbinectedin of formula (V) further comprising peaks at 2-theta angles selected from 9.2±0.2°, 12.0±0.2°, 15.2±0.2°, 18.4±0.2°, 19.2±0.2°, 20.6±0.2°, 24.9±0.2°, 26.45±0.2°.

14. A process for the preparation of polymorphic form RK-1 of Lurbinectedin of the formula (V) as claimed in claim 2, wherein the process comprises the steps of.
a) dissolution of Lurbinectedin in suitable polar solvent;
b) precipitation of polymorphic form RK-1 of Lurbinectedin by addition of suitable anti-solvent into the polar solvent specified in step a) given above or vice versa to obtain polymorphic form RK-1 of Lurbinectedin.

15. The process for the preparation of polymorphic form RK-1 of Lurbinectedin as claimed in claim 14, wherein suitable polar solvent is selected from chlorinated hydrocarbon; ester; alcohol; ketone; nitrile; amide; sulfone or mixture thereof.

16. The process for the preparation of polymorphic form RK-1 of Lurbinectedin as claimed in claim 14, wherein suitable anti-solvent is selected from hydrocarbon, chlorinated hydrocarbon, ether or mixture thereof.

17. The process for the preparation of polymorphic form RK-1 of Lurbinectedin as claimed in claimed in claim 15, wherein chlorinated hydrocarbon is selected from methylene dichloride (MDC), carbon tetrachloride, chloroform, 1, 2-dichloroethane or mixture thereof; ester is selected from ethyl acetate, isopropyl acetate, butyl acetate, isobutyl acetate, methylbutyryl acetate, ethylbutyryl acetate or Methyl isobutyrylacetate; alcohol is selected from methanol, ethanol, n-propanol or isopropanol; ketone is selected from acetone, dimethyl ketone, diethyl ketone or methyl ethyl ketone; nitrile is selected from acetonitrile, propionitrile, butyronitrile or isobutyronitrile; amide is selected from dimethylformamide (DMF), dimethylacetamide or diethyl acetamide; sulfone is selected from dimelthylsulfoxide (DMSO), or diethyl sulfone.

18. The process for the preparation of polymorphic form of RK-1 of Lurbinectedin as claimed in claim 16, wherein hydrocarbon is selected from n-pentane, n-hexane, n-heptane, n-octane. cyclohexane, benzene or toluene; chlorinated hydrocarbon is selected from chloroform or methylene dichloride (MDC); ether is selected from dimethyl ether, diethyl ether, di-isopropyl ether, methyl tert-butyl ether, tetrahydrofuran or 1,4-dioxane.

19. (canceled)

20. The process for the preparation of polymorphic form of RK-1 of Lurbinectedin as claimed in claim 14, wherein suitable anti-solvent is alkane selected from n-pentane, n-hexane, n-heptane and n-octane; preferably n-heptane.

21. The process for the preparation of polymorphic form of RK-1 of Lurbinectedin as claimed in claim 14, wherein Lurbinectedin employed in step a) is pure amorphous form of Lurbinectedin.
